(19) United States
(12) Patent Application Publication
(10) Pub. No.: US 2020/0341002 A1
(43) Pub. Date: Oct. 29, 2020

(54) METHODS FOR QUANTITATING PROTEIN BIOMARKERS

(71) Applicant: Cleveland State University, Cleveland, OH (US)

(72) Inventors: Baochuan Guo, Hudson, OH (US); Ravali Algandula, Cleveland, OH (US)

(21) Appl. No.: 16/851,478

(22) Filed: Apr. 17, 2020

Related U.S. Application Data
(60) Provisional application No. 62/835,189, filed on Apr. 17, 2019.

(51) Int. Cl. G01N 33/68; G01N 33/543; G01N 33/574

(52) U.S. Cl. CPC G01N 33/6848; G01N 2333/765; G01N 33/574; G01N 33/543

(57) ABSTRACT

Disclosed are methods of detecting biomarkers in complex biological matrices. The method comprises providing a sample well coated with antibodies specific to target protein biomarker(s), contacting the sample well with a biological sample to capture target biomarkers, digesting target biomarkers, and analyzing the signature peptides resulting from digestion of target biomarkers to determine the amount of one or more protein biomarkers by a mass spectrometry method.

### CROSS-REFERENCE

This application claims the benefit of U.S. provisional patent application Ser. No. 62/835,189, filed Apr. 17, 2019, the content of which is hereby incorporated by reference as if fully recited herein.

### FIELD

The present disclosure relates to the field of chemical characterization and quantitation. More particularly, the present disclosure relates to methods of characterizing protein biomarkers in complex biological matrices.

### BACKGROUND

Certain proteins can provide direct insight into biological activities at functional levels in an organism, these proteins are excellent candidates for biomarkers. Thereby, protein quantification has become an important tool in disease diagnosis, prognosis, and treatment. While many clinically relevant proteins exist in the μg/mL range in human plasma, most disease-related proteins and protein biomarkers fall in much lower ranges (low ng/mL or pg/mL range). Thus, there is an unmet need for reliable and accurate detection of proteins in biological matrices that can identify relevant biomarker proteins at low detection levels.

Two common analytical methods involved in protein quantitation are immunoassays and mass spectrometry (MS), but both have their drawbacks. Immunoassays, particularly enzyme linked immuno-sorbent assay (ELISA), are extensively used as clinical routine methods due to features of being fast, sensitive, and highly automatable. However, immunoassay measurements can be variable and inaccurate due to a number of factors including cross-reactivity, post-translational modifications, interference from other molecules, and the high-dose hook effect. Many commercial ELISA kits have been shown to yield variable and non-comparable absolute concentrations, leading to misleading interpretations which can result in unnecessary treatment or missed opportunities for therapeutic intervention.

On the other hand, mass spectrometry analysis only works well with purified proteins, meaning that the proteins in biological samples need to be purified prior to mass analysis.

Measurements of changes in biomarker concentration in biological samples are regularly used as a diagnostic indicator for evaluation of the degree of pathological states because such measurements help to distinguish diseases from the otherwise healthy condition. Accordingly, there is an unmet need for improved methods of determining an amount of proteinaceous biomarkers specific to diseases, especially where such biomarkers are present in complex matrices and/or are only present in low concentrations.

### SUMMARY

The general inventive concepts are based in large part on the discovery that a specific immunocapture LC-MS/MS method for protein quantitation enables accurate and reliable detection of important target proteins (i.e., biomarkers). In certain exemplary embodiments, the general inventive concepts relate to a method that combines immunocapture with mass spectrometry analysis to accurately quantitate protein biomarkers with improved selectivity and sensitivity compared to current immunoassays and mass spectrometry methods. The advantage of this inventive method is that immunocapture enriches target proteins, improving detection sensitivity, while MS analysis offers excellent specificity.

More particularly, the general inventive concepts are based on: 1) the use of 8M Urea to elute captured proteins which achieves quantitative elution of captured proteins from antibodies by breaking the bonds between antibodies and antigens; 2) in-well digestion (as opposed to solution-based digestion in a separate well) provides superior results achieving quantitative digestion; and 3) adding non-target proteins to the digestion system to enhance digestion, enables improved quantitative digestion of small-quantities of target proteins, which would otherwise not be able to quantitatively digest without an assistance of the added proteins.

In certain exemplary embodiments, the general inventive concepts relate to a method of quantitating the amount of a biomarker in a biological sample. In certain exemplary embodiments, the method comprises providing a sample well coated with antibodies specific to target proteins, contacting the sample well with a biological sample to capture target proteins, washing the well, eluting captured proteins, digesting eluted proteins and analyzing the peptides resulting from digestion of eluted proteins to determine the amount of one or more proteins by mass spectrometry.

In certain exemplary embodiments, the general inventive concepts relate to a method of determining the amount of a biomarker in a biological sample. The method comprises providing a sample well coated with an antibody specific to a target biomarker; contacting the sample well with a biological sample to form a bound sample; digesting the bound sample to form a digested sample; and analyzing the digested sample to determine the amount of one or more target biomarkers.

In certain exemplary embodiments, the general inventive concepts relate to a method of quantitating the amount of multiple biomarkers in a biological sample. In certain exemplary embodiments, the method comprises providing a sample well coated with antibodies specific to more than one target protein, contacting the sample well with a biological sample to capture the target proteins, washing the well, eluting captured proteins, digesting eluted proteins and analyzing the peptides resulting from digestion of eluted proteins to determine the amount of one or more proteins by mass spectrometry.

In certain exemplary embodiments, the general inventive concepts relate to a method of quantitating the amount of a biomarker in a biological sample. In certain exemplary embodiments, the method comprises providing a sample well having at least one antigen specific antibody coated thereon. In certain exemplary embodiments, the method comprises providing a sample well having at least one antibody specific to a protein or peptide coated thereon. In certain exemplary embodiments, the method comprises providing a sample well having at least one antibody specific to a biomarker. In certain exemplary embodiments, the antibody is specific for hemoglobin or a peptide resulting from digestion thereof.

In certain exemplary embodiments, the general inventive concepts relate to a method of determining the amount of a protein biomarker in a biological sample. The method comprises providing a sample well coated with at least one antibody specific to a target protein biomarker; contacting the sample well with a biological sample to form a bound sample; eluting the bound sample; adding a second protein to the bound sample; digesting the bound sample in the presence of urea, in a concentration of about 8M, and trypsin to form a digested sample; and analyzing the digested sample to determine the amount of one or more protein biomarkers by comparison to a tryptic peptide produced by trypsin digestion of a protein biomarker to an isotope labeled peptide standard; wherein the digested sample is analyzed by mass spectrometry.

In certain exemplary embodiments, the general inventive concepts relate to a method of determining the amount of a biomarker in a biological sample. In certain exemplary embodiments, the method comprises digestion in the presence of urea.

In certain exemplary embodiments, the general inventive concepts relate to a method of determining the amount of a biomarker in a biological sample. In certain exemplary embodiments, the method comprises addition of a second protein to the sample well prior to digestion. In certain exemplary embodiments, the second protein is bovine serum albumin.

In certain exemplary embodiments, the general inventive concepts relate to a method of determining the amount of a biomarker in a biological sample. In certain exemplary embodiments, the method comprises analysis of the digested sample by mass spectrometry.

In certain exemplary embodiments, the general inventive concepts relate to a method of determining the amount of a biomarker in a biological sample. In certain exemplary embodiments, the method comprises immunocapture and mass spectrometry analysis.

In certain embodiments, specific tryptic peptides (monitor peptides) are selected as a stoichiometric representative of target proteins, which is produced by trypsin digesting protein biomarkers. In another embodiment, the monitor peptides are quantified against a spiked internal standard (i.e. a synthetic stable-isotope labeled peptide) by mass spectrometry, yielding the protein biomarker concentration.

In certain embodiments, the antibody(s) specific to target protein biomarkers is immobilized on a solid support on a plate containing one or multiple wells. In another embodiment, the antibody(s) is immobilized on sorbents packed in a small column. In yet another embodiment, the antibody(s) is immobilized on magnetic beads. Accordingly, the instant disclosure presents novel methods to overcome the drawbacks of conventional approaches and enables determination of biomarkers to facilitate early detection and/or treatment of certain diseases.

In certain exemplary embodiments, the general inventive concepts relate to a method of screening for colorectal cancer. The method comprises providing a sample well coated with an antibody specific to hemoglobin; contacting the sample well with a stool sample to form a bound sample; digesting said bound sample to form a digested sample; and analyzing said digested sample to determine the amount of hemoglobin in the stool sample.

### BRIEF DESCRIPTION OF THE DRAWINGS

The general inventive concepts, as well as embodiments and advantages thereof, are described below in greater detail, by way of example, with reference to the drawings in which:

FIG. 1 is a schematic representation of an immunocapture-MS quantification strategy for quantitation of low abundance proteins in complex biological matrices according to the general inventive concepts.

FIG. 2 shows a general scheme for a method of determining the amount of a biomarker according to the general inventive concepts.

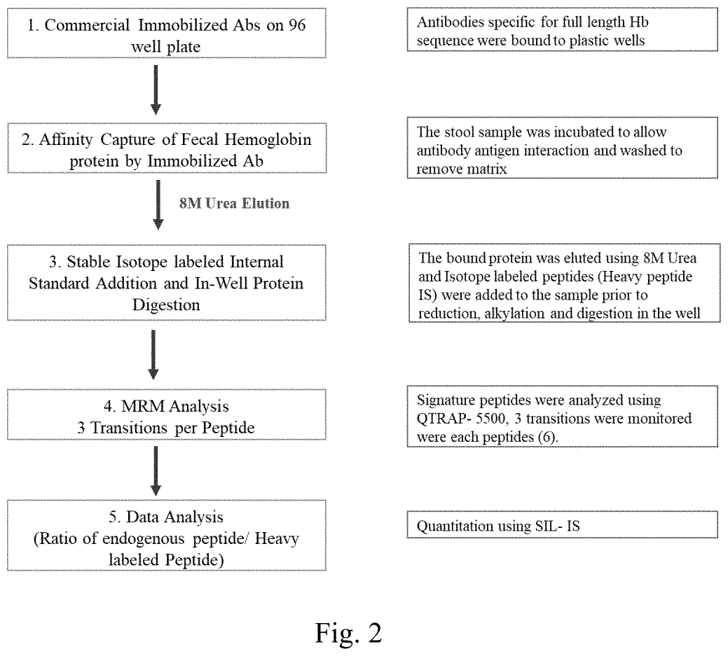

### DETAILED DESCRIPTION

The compositions and methods described herein utilize protein immunocapture enrichment along with mass spectrometry to improve quantification sensitivity for low abundance proteins. These and other features of the compositions and methods, as well as some of the many optional variations and additions, are described in detail hereafter.

The terms “mass spectrometry immunoassay”, “Immunocapture MS,” and “immunocapture mass spectrometry” are used interchangeably herein and are intended to refer to the overall general inventive concept of quantitating the amount of certain biologically important proteins and/or monitor peptides.

The term “biomarker” or “target biomarker” as used herein refers to one or more proteins, the presence of which are indicative of a condition or disease.

The term “antibody” as used herein refers one or more antibodies specific to a target biomarker for capturing a biomarker.

The term “secondary protein” as used herein refers to proteins that are not target proteins to be analyzed.

The term “monitor peptide” or “signature peptide” as used herein refers one or more peptides produced from digestion of target proteins, which is used to determine target protein concentration(s) by mass spectrometry.

The general inventive concepts are based in part on the discovery that certain protein biomarkers can be identified and isolated to allow for more accurate characterization of diseases. In certain exemplary embodiments, the method comprises providing a sample well coated with antibodies specific to target proteins, contacting the sample well with a biological sample to capture target proteins, washing the well, eluting captured proteins, digesting eluted proteins and analyzing the peptides resulting from digestion of eluted proteins to determine the amount of one or more proteins by mass spectrometry.

As previously mentioned, the general inventive concepts relate to an immunocapture mass spectrometry (MS) method for determining the amount of a biomarker in a biological sample. In certain embodiments, the method employs at least one antibody coated on solid support to capture one or more target proteins. In certain exemplary embodiments, the antibody is specific for the biomarker(s).

In certain exemplary embodiments, the general inventive concepts relate to a method of determining the amount of a biomarker in a biological sample. In certain embodiments, the method involves elution and digestion of captured proteins in the same well used to capture biomarker. In certain exemplary embodiments, the method comprises elution in the presence of urea.

In certain exemplary embodiments, the general inventive concepts relate to a method of determining the amount of a biomarker in a biological sample. In certain exemplary embodiments, the method comprises addition of a second protein to the digestion reaction prior to digestion. In certain exemplary embodiments, the second protein is bovine serum albumin.

In certain exemplary embodiments, the general inventive concepts relate to a method of determining the amount of a biomarker in a biological sample. In certain exemplary embodiments, the method comprises analysis of the digested sample by mass spectrometry.

In certain exemplary embodiments, the general inventive concepts relate to a method of determining the amount of a protein biomarker in a biological sample. The method comprises providing a sample well coated with at least one antibody specific to a target protein biomarker; contacting the sample well with a biological sample to form a bound sample; eluting the bound sample; adding a second protein to the bound sample; digesting the bound sample in the presence of urea, in a concentration of about 8M, and trypsin to form a digested sample; and analyzing the digested sample to determine the amount of one or more protein biomarkers by comparison to a tryptic peptide produced by trypsin digestion of a protein biomarker to an isotope labeled peptide standard; wherein the digested sample is analyzed by mass spectrometry.

In certain exemplary embodiments, the general inventive concepts relate to a method of screening for colorectal cancer. The method comprises providing a sample well coated with an antibody specific to hemoglobin; contacting the sample well with a stool sample to form a bound sample; digesting said bound sample to form a digested sample; and analyzing said digested sample to determine the amount of hemoglobin in the stool sample. In certain exemplary embodiments, an individual will be determined to have colorectal cancer or be at increased risk of colorectal cancer when the level of hemoglobin in the stool sample is above a predetermined standard or level. In certain exemplary embodiments, the predetermined level is based on the level found in the general population of individuals that do not have colorectal cancer.

In order to achieve the general inventive concepts, Applicants evaluated the following: 1) Determination of which procedure helps maximum target protein elution from antibody: a thorough comparison between a method that elutes target proteins first from antibody, followed by transferring eluted proteins to another tube. Then protein digestion is performed (i.e., in-solution digestion) versus another method, in which captured proteins were eluted and digested in the same well used to capture target proteins (i.e., in-well digestion); 2) Discovery that adding a secondary protein to the digestion system can lead to complete digestion of low-quantity target proteins; and 3) Development of an immunocapture mass spectrometry method to quantify hemoglobin in human stool.

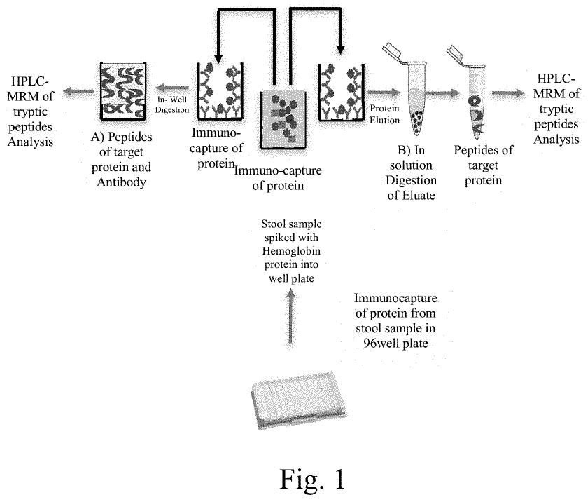

FIG. 1 shows a general schematic procedure for quantification of proteins. On the right is in-solution digestion, in which captured proteins are first eluted, followed by transferring eluted protein to another tube, where digestion is performed. Then formed peptides from digestion are analyzed to determine the amount of the target protein by mass spectrometry.

The left side of FIG. 1 shows in-well digestion according to the general inventive concepts. A biological sample is brought into contact with an antibody-coated well, the target protein is thereby captured by the antibody, the sample is washed to remove unwanted proteins etc., The captured protein is eluted and digested the same well or tube that is used to capture target proteins. Then formed peptides from digestion are removed from the well and analyzed to determine the amount of the target protein by mass spectrometry.

For the methods described herein, hemoglobin (Hb) was selected as a model (target) protein. Colorectal cancer (CRC) can be detected by the presence of Hb in stool due to bleeding. In one embodiment, the invention provides a method to quantitate human hemoglobin in stool. In another embodiment, this invention provides a method for colorectal cancer screening. In certain exemplary embodiments, the general inventive concepts contemplate the use of antibodies specific to hemoglobin to capture hemoglobin and/or peptides related to hemoglobin digestion.

As previously mentioned, the general inventive concepts contemplate an immunocapture-based sample “cleanup” approach. In certain embodiments, the method comprises an antibody-coated well. In one particular embodiment, the well comprises antibodies to capture/enrich stool hemoglobin. In certain exemplary embodiments, monitor peptides are employed as a proxy for quantification of hemoglobin. Accordingly, in certain exemplary embodiments, a method of determining amounts of a biomarker comprises digestion of a target protein using an enzyme. In certain exemplary embodiments, a method of determining a biomarker comprises digestion of a target protein is trypsin. In certain exemplary embodiments, quantification is performed using mass spectrometry. In certain exemplary embodiments, a method of determining amounts of hemoglobin comprises digestion of captured hemoglobin. In certain exemplary embodiments, the digestion enzyme is trypsin. In certain exemplary embodiments, quantification is performed using mass spectrometry.

As discussed further herein, in-well digestion can lead to an increase in the accuracy of protein quantitation. Accordingly, in certain exemplary embodiments, a method of determining a biomarker comprises in-well digestion of proteins (including target proteins).

The general inventive concepts are based, in part, on the discovery that the yield of Hb monitor peptides was higher when in-well digestion is employed in the presence of 8M Urea, as compared to in-solution digestion. Accordingly, in certain exemplary embodiments, a method of determining a biomarker comprises elution of the peptide using urea, including 8 M urea.

The general inventive concepts also contemplate use of a secondary protein to assist trypsin digestion. It was found that adding a secondary protein to the digestion system leads to better digestion of low-quantity proteins, meaning that the yield of monitor peptides was higher. For example, the LLOQ of quantifying hemoglobin dropped down to 5ng from 20 ng, a truly unexpected result. Accordingly, in certain exemplary embodiments, a method of determining the amount of proteins comprises the use of a secondary protein in digestion. In certain exemplary embodiments, the secondary protein is BSA.

A major challenge to immunocapture mass spectrometry is how to quantitatively elute and digest captured proteins. The current invention provides a method to overcome this challenge, making immunocapture mass spectrometry more accurate. Specifically, the results presented herein indicate that a combination of elution in the presence of 8M Urea, in solution digestion, and utilizing secondary proteins in digestion improved quantitation by immunocapture mass spectrometry.

The general inventive concepts provide a method to quantitatively elute and digest the target proteins captured by antibodies.

The general inventive concepts provide a method to improve trypsin digestion of low-quantity proteins by adding a secondary protein(s) to the digestion system.

The general inventive concepts discussed herein demonstrate: 1) a quantitative immunocapture platform for protein capture and enrichment, 2) quantitative elution and digestion of captured proteins in the presence of urea and secondary proteins; 3) quantitative determination of the amount of monitor peptides by mass spectrometry, thus the quantity of target proteins; and 4) application of the developed immunocapture mass spectrometry method for CRC screening.

Those of ordinary skill in the art will recognize that, while the general inventive concepts have been described with respect to inventive methods of quantitating proteins, the methods also contemplate the unique systems and compositions for such quantitating.

The following examples illustrate exemplary embodiments and/or features of the methods according to the general inventive concepts. The examples are given solely for the purpose of illustration and are not to be construed as limitations of the general inventive concepts, as many variations thereof are possible without departing from the spirit and scope of the general inventive concepts.

### EXAMPLES

In all examples, human hemoglobin was used as the model system to study immunocapture mass spectrometry. Specifically, monitor peptides produced from a and subunits of Hb were analyzed. 2 peptides (MFLSFPTTK and EFTPPVQAAYQK) were used for quantification and 5 more peptides were also monitored for evaluating immunocapture, elution and trypsin digestion. Lyophilized human hemoglobin powder was obtained from Sigma Aldrich. Quantitative Fecal Occult Blood ELISA test kit coated with anti-hemoglobin monoclonal antibodies was obtained from Epitope Diagnostics. Trypsin, 1,1-dithiothretiol, iodoacetamide, trifluoroacetic acid, and formic acid were purchased from Sigma Aldrich (St. Louis, Mo., USA). Stable isotope-labeled standards (SIS) were synthesized by New England Peptide (Gardner, Mass.) for both alpha: H2N-MFLSFPTTK-OH and beta H2N-EFTPPVQAAYQK-OH chains monitor peptides, in which, the C-terminal arginine or lysine was labeled with ¹³C and ¹⁵N. Human stool samples were collected from both healthy subjects and colorectal cancer patients.

### Example 1

This example demonstrates the benefit using urea for elution. In-well digestion worked better than in-solution digestion when used in immunocapture mass spectrometry. As previously discussed, in immunocapture mass spectrometry, captured target proteins must be eluted from antibody and digested into peptides so that mass spectrometry can quantify its monitor peptide, therefore, target protein itself. First an experiment was conducted to select an elution buffer that can effectively elute capture proteins. It was discovered that 8M urea is the best buffer among three buffers tested: 8M urea, Formic acid (FA, 0.1-2%) and trifluoroacetic acid (TFA, 0.1-1%). Table 1 summaries the study result. Clearly, the highest ion signal of monitor peptides was seen when 8M urea was used for elution.

We also compared the digestion performance of in-solution digestion (as shown in the right side of FIG. 1) and in-well elution (as shown in the left side of FIG. 1). Table 1 summaries the result of this comparison study. Clearly, the higher ion signal was seen when in-well digestion was used.

TABLE 1

| | 8M Urea | | 8M Urea | | 0.5% FA | | 9.5% FA | | 0.5% TFA | | 0.5% TFA | |
|---|---|---|---|---|---|---|---|---|---|---|---|---|
| Signature | in-well | | in-solution | | in-well | | Elute- | | in-well | | Elute- | |
| Peptide | digestion | | digestion | | digestion | | digestion | | digestion | | digestion | |
| 50 ng Hb | Area | % RSD | Area | % RSD | Area | % RSD | Area | % RSD | Area | % RSD | Area | % RSD |
| α MFLSFPTTK | 6.0*10⁴ | 2.5 | 4.0*10⁴ | 1.97 | 2*10² | 1.3 | 1.0*10² | 4.12 | 1.0*10² | 0.84 | NA | NA |
| (5336.3+ + −593.3) | | | | | | | | | | | | |
| β | 4.5*10⁴ | 3.2 | 1.5*10⁴ | 0.9 | 0.9*10² | 2.4 | 1.0*10² | 3.40 | 0.9*10² | 1.20 | NA | NA |
| EFTPPVQAAYQK | | | | | | | | | | | | |
| (690.1+ + −807.5) | | | | | | | | | | | | |

### Example 2

In this study, it was discovered that adding BSA (a secondary protein) to the digestion system significantly improved trypsin digestion of low-quantity hemoglobin. Applicants assessed how to further improve immunocapture mass spectrometry to enhance target protein quantitation by mass spectrometry after achieving quantitative elution of target peptides using 8M Urea over formic acid and acetic acid.

It was surprisingly discovered that addition of 2 μg bovine serum albumin (BSA) (e.g., a secondary protein) just before adding trypsin to the antibody coated well in the presence of captured hemoglobin using 100 ng trypsin significantly improved the overall digestion efficiency. The digestion in the presence of BSA gave much stronger monitor peptide signals, decreasing the LOD for hemoglobin quantitation to 1.0 ng. This was confirmed by comparing the peak areas of peptides obtained in the presence or absence of BSA in the digestion system. Accordingly, in certain exemplary embodiments, a method of quantitating a biomarker comprises addition of a second protein. In certain exemplary embodiments, the protein is bovine serum albumin (BSA). Table 3 shows the peak areas of Hb monitor peptides in the presence of various amounts of BSA added to the digestion systems.

TABLE 3

| | α Chain Monitor Peptide Signal | | | | β Chain Monitor Peptide Signal | | | |
|---|---|---|---|---|---|---|---|---|
| Conc. BSA | MFLSFPTTK | | | | EFTPPVQAAYQK | | | |
| (ug) | NO BSA | 0.1 ug | 1 ug | 2 ug | NO BSA | 0.1 ug | 1 ug | 2 ug |
| Replicate 1 | 450 | 5000 | 10500 | 20000 | 320 | 4500 | 9500 | 12500 |
| Replicate 2 | 600 | 4570 | 12000 | 18400 | 390 | 3940 | 8990 | 11000 |
| Replicate 3 | 490 | 3940 | 10300 | 19200 | 410 | 3600 | 8500 | 9710 |
| Avg Signal | 513 | 4503 | 10933 | 19200 | 373 | 4013 | 8996 | 11070 |

Several additional experiments were conducted to check for any cross talk or interference peaks from BSA that was eluting at the same retention time of Hb monitor peptides. We found that none of the detection channels had any cross talk from peptides produced from BSA digestion, proving that an increased signal in the presence of BSA is not due to co-elution of any BSA digestion peptides. The studies suggested that the presence of BSA as a secondary protein significantly improved the efficiency of trypsin digestion of low-quantity proteins.

### Example 3

This example demonstrates the utility of using our immunocapture mass spectrometry assay to quantitate human hemoglobin in stool for colorectal cancer screening. First, the performance characteristics of immunocapture of Hb in solvent vs stool was examined. For immunocapture mass spectrometry to be applied clinically, the assay must be capable of precise and accurate measurements of Hb in stool. To determine the performance characteristics of our immunocapture system, Applicants assessed the linear range, limit of quantification, accuracy, and precision for our model antigen, Hb. Known concentrations of Hb was spiked in both blank water and stool and incubated in antibody well, and the antibody capture efficiency was evaluated by comparing the peak areas of monitor peptides in water against stool, using SIL-IS spiked after capture and before digestion. It was found that the monitor peptide peak area in water was almost the same as that as in stool, demonstrating that even in the presence of the stool matrix, the antibody was specific enough to capture target Hb protein. These results were further confirmed by additional ELISA studies. These results demonstrated that the immunocapture system used to capture Hb was highly efficient and reliable in the presence of harsh matrix like stool.

The quantitation linearity of the assay was also tested at 5, 10, 20, 50, 100, 150 and 200 ng/100 μL Hb spiked in blank pooled stool (n=3). Overall, the response for monitor peptides was linear (R²=0.993 and 0.994) over about 2.5 orders of magnitude with limits of quantification around 90 fmol (˜1.5 ng) for Hb alpha chain monitor peptide and 123 fmol (˜2.0 ng) for beta chain monitor peptide.

In addition, repeatability (n=5), accuracy (n=3) and precision (n=3) were evaluated at 3 QC concentrations (12, 40 and 160 ng) along with calibration standards. Overall, the % CVs and % RE for the three QC levels are excellent and range from 8% to 14%. LOD (1.5 ng Hb) was also determined in samples spiked with Hb.

After establishing that our immunocapture mass spectrometry assay was accurate and reliable, we applied it to detect human Hb in stool samples that have been analyzed by clinical ELISA assay. 10 Hb positive stool samples collected from confirmed CRC patients and 10 Hb negative samples collected from confirmed normal subjects without CRC were used in this study. Except for one Hb positive sample, all other 19 samples were correctly identified by our immunocapture mass spectrometry assay. One sample that was not identified was later re-analyzed by ELISA, that could not detect Hb as well, suggesting that Hb in this stool sample may have been degraded because it has been collected for over 3 years.

Accordingly, the instant disclosure demonstrates: 1) the use of 8M Urea to elute captured proteins which achieves quantitative elution of captured proteins from anti-bodies by breaking the bonds between antibodies and antigens; 2) in-well digestion (as opposed to solution-based digestion) provides superior results achieving quantitative digestion; and 3) adding non-target proteins to the digestion system to enhance digestion, enables improved quantitative digestion of small-quantities of proteins, which would otherwise not be able to quantitatively digest without an assistance of the added proteins; and 4) an immunocapture mass spectrometry method for quantifying human hemoglobin in stool, thus colorectal cancer screening.

Thus, the general inventive concepts relate to a method of quantitating the amount of a biomarker in a biological sample. In certain exemplary embodiments, the method comprises providing a sample well, contacting the sample well with a biological sample, digesting the biological sample, washing the digested sample, and analyzing the resulting digested sample to determine an amount of one or more biomarkers.

Numerical ranges as used herein are intended to include every number and subset of numbers within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All references to singular characteristics or limitations of the general inventive concepts shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The compositions and methods may comprise, consist of, or consist essentially of the essential elements of the compositions and methods as described herein, as well as any additional or optional element described herein or otherwise useful in protein quantitation applications.

Unless otherwise indicated herein, all sub-embodiments and optional embodiments are respective sub-embodiments and optional embodiments to all embodiments described herein. While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative compositions or formulations, and illustrative examples shown and described.

Accordingly, departures may be made from such details without departing from the spirit or scope of the general disclosure herein.

What is claimed is:

1. A method of determining the amount of a biomarker in a biological sample, the method comprising:
providing a sample well coated with an antibody specific to a target biomarker;
contacting the sample well with a biological sample to form a bound sample;
digesting the bound sample to form a digested sample;
and analyzing the digested sample to determine the amount of one or more target biomarkers.

2. The method of claim 1, wherein the target biomarker is a protein.

3. The method of claim 2, wherein digesting the bound sample comprises digesting the sample to produce one or more signature peptides from the protein.

4. The method of claim 1, wherein the sample well is coated with antibodies specific to more than one target biomarker.

5. The method of claim 1, further comprising washing the well after contacting the well with the biological sample.

6. The method of claim 1, wherein the target biomarker is captured by the antibodies.

7. The method of claim 2, wherein the target biomarker comprises more than one protein and digesting the bound sample comprises digesting the sample to produce one or more signature peptides for each protein.

8. The method of claim 1, wherein the amount of one or more target biomarkers is determined by mass spectrometry.

9. The method of claim 1, wherein the antibody is specific for hemoglobin or a peptide resulting from digestion thereof.

10. The method of claim 2, wherein a second protein is added to the sample well prior to digestion.

11. The method of claim 11, wherein the second protein is bovine serum albumin.

12. The method of claim 1, wherein the method comprises digesting the bound sample in the presence of urea.

13. The method of claim 1, wherein the amount of one or more target biomarkers is determined by comparison to an internal standard.

14. The method of claim 13, wherein the internal standard is a peptide.

15. The method of claim 13, wherein the internal standard peptide comprises an isotope labeled peptide.

16. A method of determining the amount of a protein biomarker in a biological sample, the method comprising:
providing a sample well coated with at least one antibody specific to a target protein biomarker;
contacting the sample well with a biological sample to form a bound sample;
eluting the bound sample;
adding a second protein to the bound sample;
digesting the bound sample in the presence of urea in a concentration of about 8M and trypsin to form a digested sample;
and analyzing the digested sample to determine the amount of one or more protein biomarkers by comparison to a tryptic peptide produced by trypsin digestion of a protein biomarker to an isotope labeled peptide standard; wherein the digested sample is analyzed by mass spectrometry.

17. The method of claim 16, wherein the second protein is bovine serum albumin.

18. A method of screening for colorectal cancer, the method comprising:
providing a sample well coated with an antibody specific to hemoglobin;
contacting the sample well with a stool sample to form a bound sample;
digesting said bound sample to form a digested sample;
and analyzing said digested sample to determine the amount of hemoglobin in the stool sample.

19. The method of claim 18, wherein digesting the bound sample comprises digesting the sample to produce one or more signature peptides from hemoglobin.

20. The method of claim 18, further comprising washing the well after contacting the well with the stool sample.

21. The method of claim 18, wherein said hemoglobin is captured by the antibodies.

22. The method of claim 18, wherein the amount of hemoglobin is determined by mass spectrometry.

23. The method of claim 18, wherein a second protein is added to the sample well prior to digestion.

24. The method of claim 23, wherein the second protein is bovine serum albumin.

25. The method of claim 18, wherein the amount of hemoglobin is determined by comparison to an internal peptide standard.

26. The method of claim 25, wherein the internal peptide standard is an isotope labeled peptide.

27. The method of claim 18, wherein the presence of colorectal tumor is determined by the amount of hemoglobin determined.
